(19) United States
(12) Patent Application Publication
(10) Pub. No.: US 2025/0195861 A1
(43) Pub. Date: Jun. 19, 2025

(54) CATHETER HAVING HYPOTUBES JOINED BY LINK MEMBER AND METHOD OF MAKING THE SAME

(71) Applicants: STRYKER CORPORATION, Kalamazoo, MI (US); STRYKER EUROPEAN OPERATIONS LIMITED, Carrigtwohill (IE)

(72) Inventors: Jeffrey Wayne Baxter, San Jose, CA (US); Michael Troy Poor, San Jose, CA (US)

(73) Assignees: STRYKER CORPORATION, Kalamazoo, MI (US); STRYKER EUROPEAN OPERATIONS LIMITED, Carrigtwohill (IE)

(21) Appl. No.: 19/067,684

(22) Filed: Feb. 28, 2025

Related U.S. Application Data
(63) Continuation of application No. PCT/US2023/071673, filed on Aug. 4, 2023.
(60) Provisional application No. 63/404,383, filed on Sep. 7, 2022.

(51) Int. Cl. A61M 39/10; A61M 25/00

(52) U.S. Cl. CPC A61M 39/10; A61M 25/0013; A61M 25/0045

(57) ABSTRACT

A catheter includes: a first tubular structure; a second tubular structure, wherein the first tubular structure and the second tubular structure are arranged serially with respect to each other along a longitudinal axis of the catheter; and a polymeric connector located between (1) a part of the first tubular structure at an end of the first tubular structure and (2) a part of the second tubular structure at an end of the second tubular structure; wherein the polymeric connector is configured to couple the first tubular structure and the second tubular structure to each other, and/or to prevent the first tubular structure and the second tubular structure from separating from each other.

RELATED APPLICATION DATA

The application is a continuation of International Patent Application No. PCT/US2023/071673, filed on Aug. 4, 2023, which claims the benefit of U.S. Provisional Patent Application Ser. No. 63/404,383, filed on Sep. 7, 2022, the entire disclosures of all of which are hereby incorporated herein by reference in their entirety into the present application.

### FIELD

The present disclosure relates generally to medical devices, and more specifically to catheters and methods of making catheters.

### BACKGROUND

The use of catheters, such as intravascular catheters, for accessing and treating various types of diseases is well-known. For example, a suitable intravascular catheter may be inserted into the vascular system of a patient. Commonly used vascular application to access a target site in a patient involves inserting a guidewire through an incision in the femoral artery near the groin, and advancing the guidewire until it reaches the target site. Then, a catheter is advanced over the guidewire until an open distal end of the catheter is disposed at the target site. Simultaneously or after placement of the distal end of the catheter at the target site, an intravascular implant is advanced through the catheter via a delivery wire.

In certain applications, such as neurovascular treatment, the catheters are required to navigate tortuous and intricate vasculature. By using an appropriately sized catheter having the requisite performance characteristics, such as “pushability” “steerability”, “torquability” and most important, distal tip flexibility, virtually any target site in the vascular system may be accessed, including that within the tortuous cerebral and peripheral vasculature. Further, the forces applied at the proximal end of these catheters should be transferred to the distal ends for suitable pushability (axial rigidity) and torqueability (rotation). Achieving a balance between these features is highly desirable, but difficult. For example, a catheter may comprise variable stiffness sections (e.g., achieved by varying ratio of material, including selective reinforcement) suitable to provide sufficient flexibility, kink resistance, pushability, and torqueability to allow navigation through vasculature.

In some cases, catheters may include slots along their elongated body or selected portions thereof. Incorporating slots into these elongated medical devices can modify or customize the device flexibility/stiffness. For example, distal portions of catheters, guidewires or other delivery members may have a slot pattern (e.g., more slots per area, longer slots, and/or wider slots) that increases the flexibility thereof. When used as components of a delivery system, the slotted elongated tubular devices are preferably substantially sealed (e.g., using sheath, jacket, coating or their like), in order to prevent fluid exchange (into or out of) the inner lumen of the tube, and also to enhance lubricity. Exemplary slotted and coated medical devices are disclosed in U.S. Pat. Nos. 5,095,915, 5,443,455, 6,488,637, and 9,162,040, the entire disclosures of which are incorporated by reference herein.

### SUMMARY

A catheter includes: a first tubular structure; a second tubular structure, wherein the first tubular structure and the second tubular structure are arranged serially with respect to each other along a longitudinal axis of the catheter; and a polymeric connector located between (1) a part of the first tubular structure at an end of the first tubular structure and (2) a part of the second tubular structure at an end of the second tubular structure; wherein the polymeric connector is configured to couple the first tubular structure and the second tubular structure to each other, and/or to prevent the first tubular structure and the second tubular structure from separating from each other.

Optionally, the first tubular structure comprising a first opening at an end of the first tubular structure; the second tubular structure comprising a first opening at an end of the second tubular structure; and the polymeric connector comprises a first connector part disposed in the first opening at the end of the first tubular structure, and a second connector part disposed in the first opening at the end of the second tubular structure.

Optionally, the first opening at the end of the first tubular structure is a laser-cut opening.

Optionally, the first tubular structure comprises a protrusion at the end of the first tubular structure; the second tubular structure comprises an opening at the end of the second tubular structure, the opening sized to accommodate at least a part of the protrusion; and the polymeric connector is located in a part of the opening to prevent the protrusion of the first tubular structure from moving within the opening.

Optionally, the first tubular structure is a first hypotube made from a metal, an alloy, or a polymer, and wherein the second tubular structure is a second hypotube made from the metal, the alloy, the polymer, another metal, another alloy, or another polymer.

Optionally, the catheter further includes a liner, wherein the liner is on a first inner surface of the first tubular structure, and on a second inner surface of the second tubular structure, and wherein the polymeric connector is connected to the liner.

Optionally, the catheter further includes a jacket, wherein the jacket is on a first outer surface of the first tubular structure, and on a second outer surface of the second tubular structure, and wherein the polymeric connector is connected to the jacket.

Optionally, the jacket and at least a part of the polymeric connector are made from a same material.

Optionally, the liner and at least a part of the polymeric connector are made from a same material.

Optionally, the first tubular structure and the second tubular structure are made from different respective materials.

Optionally, the first tubular structure and the second tubular structure have different respective inner diameters and/or different respective outer diameters.

Optionally, the first tubular structure and the second tubular structure have a same inner diameter and/or a same outer diameter.

Optionally, the polymeric connector is configured to transfer one or more forces between the first tubular structure and the second tubular structure, and wherein the one or more forces comprise a compressive force, a tensile force, a shear force, a torsional force, or any combination of the foregoing.

Optionally, the end of the first tubular structure and the end of the second tubular structure are connected to each other without welding, without soldering, and without utilizing any adhesive.

Optionally, the polymeric connector has a first enlarged portion at a first end of the polymeric connector, and a second enlarged portion at a second end of the polymeric connector.

Optionally, the polymeric connector has a dumbbell shape or an I-shape.

Optionally, the catheter further includes an additional polymeric connector, wherein the additional polymeric connector is configured to couple the first tubular structure and the second tubular structure to each other, and/or prevent the first tubular structure and the second tubular structure from separating from each other.

Optionally, the polymeric connector and the additional polymeric connector are arranged circumferentially around the longitudinal axis of the catheter, and are located in a plane that is perpendicular to the longitudinal axis of the catheter.

Optionally, the polymeric connector and the additional polymeric connector are in different respective planes that are perpendicular to the longitudinal axis of the catheter.

Optionally, the polymeric connector and the second polymeric connector are parts of a filler structure that has a tubular or ring-like configuration.

Optionally, each of the first and second tubular structures comprises a structural component, wherein the structural component of the first tubular structure is configured to abut and mate with the structural component of the second tubular structure, and wherein the structural components are configured to transmit shear force.

A method of making a part of a catheter, includes: providing a first tubular structure; providing a second tubular structure; arranging the first tubular structure and the second tubular structure serially with respect to each other; and forming a polymeric connector in a space between an end of the first tubular structure and an end of the second tubular structure, wherein the formed polymeric connector couples the first tubular structure and the second tubular structure to each other, and/or prevents the first tubular structure and the second tubular structure from separating from each other.

Optionally, in the method, the first tubular structure comprising a first opening at an end of the first tubular structure; the second tubular structure comprising a first opening at an end of the second tubular structure; and the formed polymeric connector comprises a first connector part disposed in the first opening at the end of the first tubular structure, and a second connector part disposed in the first opening at the end of the second tubular structure.

Optionally, in the method, the first opening at the end of the first tubular structure is a laser-cut opening.

Optionally, in the method, the first tubular structure comprises a protrusion at the end of the first tubular structure; the second tubular structure comprises an opening at the end of the second tubular structure, the opening sized to accommodate at least a part of the protrusion; and the formed polymeric connector is located in a part of the opening to prevent the protrusion of the first tubular structure from moving within the opening.

Optionally, in the method, the first tubular structure is a first hypotube made from a metal, an alloy, or a polymer, and wherein the second tubular structure is a second hypotube made from the metal, the alloy, the polymer, another metal, another alloy, or another polymer.

Optionally, in the method, a thickness of the first tubular structure is equal to a thickness of the second tubular structure.

Optionally, in the method, a thickness of the first tubular structure is different from a thickness of the second tubular structure.

Optionally, in the method, a material of the first tubular structure is the same as a material of the second tubular structure.

Optionally, in the method, a material of the first tubular structure is different from a material of the second tubular structure.

Optionally, in the method, the formed polymeric connector has a first enlarged portion at a first end of the polymeric connector, and a second enlarged portion at a second end of the polymeric connector.

Optionally, in the method, the polymeric connector has a dumbbell shape or an I-shape.

Optionally, the method further includes providing a liner, wherein the liner is on a first inner surface of the first tubular structure, and on a second inner surface of the second tubular structure, and wherein the polymeric connector is connected to the liner.

Optionally, the method further includes providing a jacket, wherein the jacket is on a first outer surface of the first tubular structure, and on a second outer surface of the second tubular structure, and wherein the polymeric connector is connected to the jacket.

Optionally, the act of providing the liner, the act of providing the jacket, and the act of forming the polymeric connector comprise: placing the liner inside the first and second tubular structures, placing a material of the polymeric connector in the space between the end of the first tubular structure and the end of the second tubular structure, placing the jacket around the first and second tubular structures, and curing the material of the polymeric connector to form the polymeric connector.

Optionally, in the method, the liner is made from a liner material, and the act of forming the polymeric connector comprises letting some of the liner material flow into the space that is between the end of the first tubular structure and the end of the second tubular structure.

Optionally, in the method, the jacket is made from a jacket material, and the act of forming the polymeric connector comprises letting some of the jacket material flow into the space that is between the end of the first tubular structure and the end of the second tubular structure.

Optionally, in the method, the liner material flows from a liner side to a jacket side. Optionally, in the method, the jacket material flows from a jacket side to a liner side.

Optionally, in the method, the liner material flows from a liner side, and the jacket material flows from a jacket side, and the liner material and the jacket material meets at a location somewhere in the space.

Optionally, the act of providing the liner, the act of providing the jacket, and the act of forming the polymeric connector comprise: dipping the first and second tubular structures into a material to coat the first and second tubular structures with the material, and curing the material to form the liner, the jacket, and the polymeric connector.

Optionally, the act of providing the liner, the act of providing the jacket, and the act of forming the polymeric connector comprise: spraying the first and second tubular structures with a material to coat the first and second tubular structures with the material, and curing the material to form the liner, the jacket, and the polymeric connector.

Optionally, the act of forming the polymeric connector comprise: laminating a part of the liner and a part of the jacket across the space to create the polymeric connector.

Optionally, in the method, the jacket and at least a part of the polymeric connector are made from a same material.

Optionally, in the method, the liner and at least a part of the polymeric connector are made from a same material.

Optionally, in the method, the first tubular structure and the second tubular structure have different respective inner diameters and/or different respective outer diameters.

Optionally, in the method, the first tubular structure and the second tubular structure have a same inner diameter and/or a same outer diameter.

Optionally, in the method, the formed polymeric connector is configured to transfer one or more forces between the first tubular structure and the second tubular structure, and wherein the one or more forces comprise a compressive force, a tensile force, a shear force, a torsional force, or any combination of the foregoing.

Optionally, in the method, the end of the first tubular structure and the end of the second tubular structure are connected to each other without welding, without soldering and without utilizing any adhesive.

Optionally, the method further includes forming an additional polymeric connector, wherein the additional polymeric connector is configured to couple the first tubular structure and the second tubular structure to each other, and/or prevent the first tubular structure and the second tubular structure from separating from each other.

Optionally, in the method, the polymeric connector and the additional polymeric connector are arranged circumferentially around the longitudinal axis of the catheter, and are located in a plane that is perpendicular to the longitudinal axis of the catheter.

Optionally, in the method, the polymeric connector and the additional polymeric connector are in different respective planes that are perpendicular to the longitudinal axis of the catheter.

Optionally, in the method, the polymeric connector and the second polymeric connector are parts of a filler structure that has a tubular or ring-like configuration.

Optionally, in the method, each of the first and second tubular structures comprises a structural component, wherein the structural component of the first tubular structure is configured to abut and mate with the structural component of the second tubular structure, and wherein the structural components are configured to transmit shear force.

Other and further aspects and features of embodiments will become apparent from the ensuing detailed description in view of the accompanying figures.

### BRIEF DESCRIPTION OF THE FIGURES

FIG. 1 illustrates a catheter;

FIG. 2 illustrates a section of the catheter of FIG. 1, particularly showing the section having two tubular structures connected to each other;

FIG. 3A illustrates an example of the tubular structures before they are connected;

FIG. 3B illustrates the tubular structures of FIG. 3A after they are connected;

FIGS. 4A-4C illustrate examples of polymeric connectors for connecting two tubular structures;

FIG. 5 illustrates exemplary forces being transferred by a polymeric connector connecting two tubular structures;

FIG. 6 illustrates a side view of a section of the catheter of FIG. 1, particularly showing the polymeric connector being integral with a liner and a jacket;

FIG. 7 illustrates a side view of a section of the catheter of FIG. 1, particularly showing the polymeric connector formed by two connector portions;

FIG. 8 illustrates another example of a catheter having polymeric connectors connecting two tubular structures;

FIG. 9 illustrates another example of a catheter having polymeric connectors connecting two tubular structures;

FIG. 10 illustrates a method of making a part of a catheter.

### DETAILED DESCRIPTION

Various embodiments are described hereinafter with reference to the figures. It should be noted that the figures are not drawn to scale and that elements of similar structures or functions are represented by the same reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

New catheters and new techniques of making catheters are described herein. An exemplary catheter includes at least two tubular structures arranged serially along a longitudinal axis of the catheter. The two tubular structures are aligned circumferentially with respect to each other, and are connected to each other via one or more polymeric connectors. Using the polymeric connectors to connect the two tubular structures are advantageous because they allow the two tubular structures to be connected, regardless of whether the two tubular structures are made from the same material or different materials (e.g., nitinol+stainless steel, nitinol+platinum iridium, different polymers, etc.), and/or regardless of whether the two tubular structures have the same internal diameter, different internal diameters, the same outer diameter, or different internal diameters. This is advantageous over tubular connections utilizing welding (which may not work when the tubular structures are made from dissimilar materials) or glue (which may be messy to implement, and may not provide desirable structural and/or dimensional characteristics). The polymeric connectors described herein are also advantageous because they may be configured to be compliance in one or more degrees of freedom.

In one exemplary technique described herein, a cut pattern is formed at an end of a first tubular structure (e.g., first hypotube), and a cut pattern is formed at an end of a second tubular structure (e.g., second hypotube). The two ends of the respective tubular structures are aligned so the cut patterns at the respective ends of the respective tubular structures are ready to be connected. A polymeric jacket is placed over the two tubular structures, and a polymeric liner is placed within the two tubular structures, thereby creating a configuration in which the two tubular structures are sandwiched between the polymeric jacket and the polymeric liner. The polymeric jacket and/or the polymeric liner is then heated to cause some polymeric material (from the jacket and/or from the liner) to flow into the cut patterns at the ends of the tubular structures and/or into a space between the ends of the tubular structures. Once the polymeric material is cooled/cured, the polymeric connector is then formed.

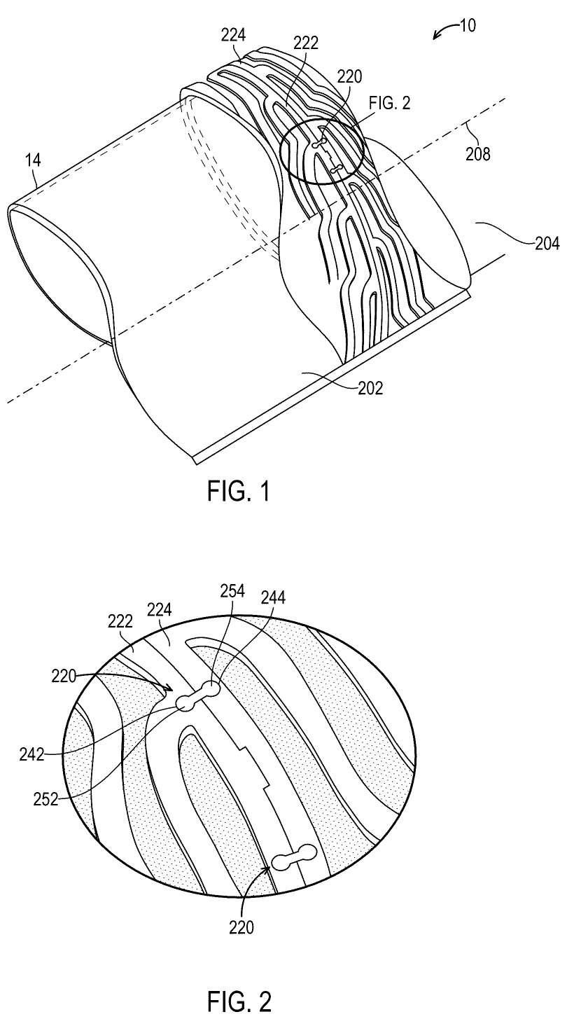

FIG. 1 depicts a catheter 10 according to some embodiments. The catheter 10 is configured for insertion into a blood vessel. The catheter 10 may be a delivery catheter configured to deliver an item (e.g., implants (such as coils, stents, etc), drugs, diagnostic agent, etc.) into a human body and/or to retrieve an item (e.g., blood, biopsy, etc.) from the human body for delivery outside of the human body, a treatment catheter comprising an energy delivery device configured to provide treatment, a diagnostic catheter configured to image an internal part of a human body, or any of other types of catheter (e.g., a balloon catheter).

The catheter 10 includes an elongated member 11 having a proximal end 15, a distal end 16, and a body 13 extending between the proximal end 15 and the distal end 16. The elongated member 11 comprises a tubular member 14. The tubular member 14 is configured to provide certain structural characteristic(s) along a longitudinal axis of the elongated member 11. The tubular member 14 may be any elongated device or component having a lumen that can be formed from any material, such as, any suitable biocompatible metal, polymer, or a combination thereof. The tubular member 14 may have a circular cross-section in some embodiments. In other embodiments, the tubular member 14 of the elongated member 11 may have any cross-sectional shapes, such as an elliptical shape, or any custom-design shape. Also, in some embodiments, the tubular member 14 may have different cross-sectional shapes and/or cross-sectional dimensions along a longitudinal axis of the tubular member 14.

The tubular member 14 may have a wall thickness that is anywhere between 0.001 inch and 0.01 inch, or anywhere between 0.001 inch and 0.006 inch, or anywhere between 0.002 inch and 0.004 inch. In other embodiments, the tubular member 14 may have a wall thickness that is higher than 0.01 inch or less than 0.001 inch. In some cases, the tubular member 14 may have an outer diameter that is anywhere from 0.01 inch to 0.11 inch, or anywhere from 0.08 inch to 0.11 inch, or anywhere from 0.01 inch to 0.03 inch (e.g., 0.024 inch +/−0.002 inch), or anywhere from 0.01 inch to 0.02 inch. In other cases, the tubular member 14 may have an outer diameter that is more than 0.11 inch. In further cases, the tubular member 14 may have an outer diameter that is less than 0.01 inch.

As shown in FIG. 2, the tubular member 14 of the catheter 10 includes a first tubular structure 202 and a second tubular structure 204, wherein the first tubular structure 202 and the second tubular structure 204 are arranged serially with respect to each other along a longitudinal axis 208 of the catheter 10. The tubular member 14 of the catheter 10 also includes a polymeric connector 220 located between (1) a part of the first tubular structure 202 at an end 222 of the first tubular structure 202 and (2) a part of the second tubular structure 204 at an end 224 of the second tubular structure 204. The polymeric connector 220 is configured to couple the first tubular structure 202 and the second tubular structure 204 to each other, and/or to prevent the first tubular structure 202 and the second tubular structure 204 from separating from each other.

As shown in FIG. 2, in some embodiments, the first tubular structure 202 comprises a first opening 242 at the end 222 of the first tubular structure 202, and the second tubular structure 204 comprises a first opening 244 at the end of 224 the second tubular structure 204. The polymeric connector 220 comprises a first connector part 252 disposed in the first opening 242 at the end 222 of the first tubular structure 202, and a second connector part 254 disposed in the first opening 244 at the end 224 of the second tubular structure 204.

In some embodiments, the first opening 242 at the end 222 of the first tubular structure 202 is a laser-cut opening. Also, in some embodiments, the first opening 244 at the end 224 of the second tubular structure 204 is a laser-cut opening. In other embodiments, one or both of the openings 242, 244 may be created by mechanical cutting, hole-punching, chemical etching, etc.

In some embodiments, the first tubular structure 202 may be a first hypotube made from a metal, an alloy, or a polymer, and the second tubular structure 204 may be a second hypotube made from the metal, the alloy, the polymer, another metal, another alloy, or another polymer.

Also, in some embodiments, the first tubular structure 202 may have a plurality of slots, openings, etc. forming a porous wall for the first tubular structure 202. Similarly, in some embodiments, the second tubular structure 204 may have a plurality of slots, openings, etc., forming a porous wall for the second tubular structure 204. By means of non-limiting examples, each of the first tubular structure 202 and the second tubular structure 204 may be a tube with laser-cut openings, a braided tubular structure, a wire mesh, a coil, a tube with slots/gaps formed by laser-cutting or machine-cutting, etc.

In some embodiments, the openings/slots/gaps in each of the first and second tubular structures 202, 204 may be formed by saw-cutting the tubular structure 202/204 with circular blades, micro-machining, laser cutting, electric discharge machining, plasma arc cutting, grinding, milling, casting, molding, chemically etching, or other known suitable methods, and the like. In other embodiments, the tubular structure 202/204 with the openings/slots/gaps may be produced using “additive” manufacturing (e.g., 3D printing) rather than the various “subtractive” techniques described.

In some cases, the first tubular structure 202 may have multiple first openings 242 at the end 222 of the first tubular structure 202, and the second tubular structure 204 may have multiple first openings 244 at the end 224 of the second tubular structure 204. The first openings 242 of the first tubular structure 202 are configured to align respectively with the first openings 244 of the second tubular structure 204, wherein each pair of aligned first openings 242 form a space for accommodating a corresponding polymeric connector 220.

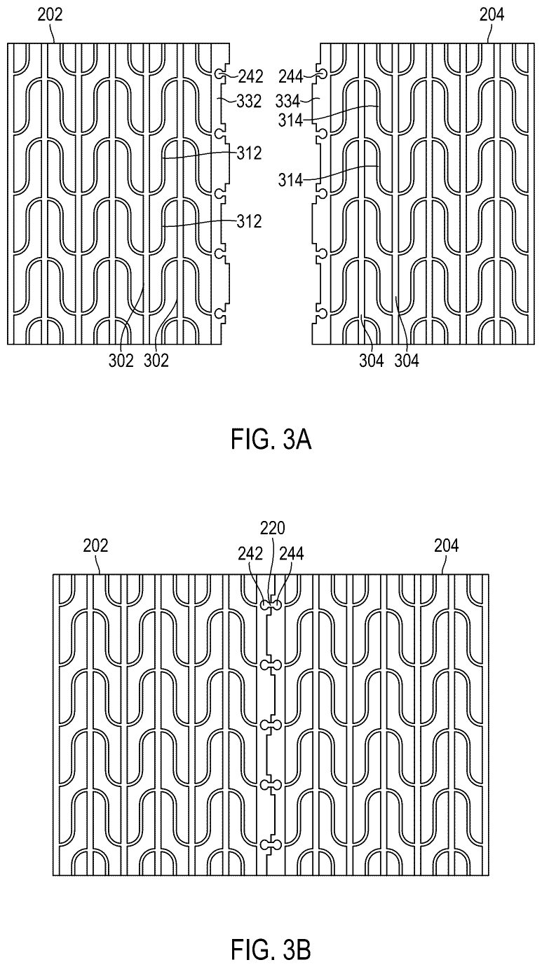

FIG. 3A illustrates an example of the first tubular structure 202, particularly showing the first tubular structure 202 having multiple ring elements 302 arranged in series along a longitudinal axis of the first tubular structure 202. Adjacent ring elements 302 are connected by a number of structural members 312. The ring elements 302 and the structural members 312 cooperate together to transmit one or more forces during use of the catheter 10. By means of non-limiting examples, the one or more forces may be axial compression, axial tension, bending, shear, torsion, or any combination of the foregoing.

Similarly, the second tubular structure 204 has multiple ring elements 304 arranged in series along a longitudinal axis of the second tubular structure 204. Adjacent ring elements 304 are connected by a number of structural members 314. The ring elements 304 and the structural members 314 cooperate together to transmit one or more forces during use of the catheter 10. By means of non-limiting examples, the one or more forces may be axial compression, axial tension, bending, shear, torsion, or any combination of the foregoing.

The openings defined by the ring elements and structural members of the tubular structures 202, 204 are advantageous because they enhance the flexibility of the tubular member 14 of the catheter 10, while the structural members (e.g., beams) and ring elements (annular segments) provide a relatively high degree of torsional stiffness, while retaining a desired level of lateral bending flexibility.

It should be noted that the tubular structure 202/204 is not limited to having the configuration and features described in the above examples, and that the tubular structure 202/204 may have other configurations and features in other embodiments. For example, in other embodiments, each of the tubular structure 202, 204 may have different arrangements and configurations of ring elements and structural members.

In some embodiments, at least some or all of the structural members are disposed such that their respective longitudinal axes form a same angle or similar angles (e.g., 0 degrees +/−10 degrees) with the longitudinal axis of the catheter 10. In other embodiments, the structural members are disposed such that their respective longitudinal axes form different angles with the longitudinal axis of the catheter 10. It should be appreciated that the distribution and/or configuration of the ring elements and structural members may have any suitable variations and combinations thereof.

Also, in some cases, the tubular structures 202, 204 with the cut pattern may provide one or more mechanical requirements (e.g., axial stiffness, hoop strength, bending stiffness, bending radius, torsional stiffness, etc., or any combination of the foregoing).

In addition, in some cases, it may be desirable to provide the proximal portion of the catheter 10 with higher stiffness. Such may be implemented using tubular structures 202, 204 with different geometrical, mechanical, and/or material properties (e.g., different thicknesses, different materials, different tube opening patterns, etc.).

As shown in FIG. 3A, the first tubular structure 202 has multiple first openings 242 at the end of the first tubular structure 202, and the second tubular structure 204 has multiple first openings 244 at the end of the second tubular structure 204. Also as shown in the figure, the first tubular structure 202 also has a structural component 332 (e.g., tube portion with opening) configured to abut and mate with a corresponding structural component 334 (e.g., protrusion) at the end of the second tubular structure 204. The structural components 332, 334 form an indexing element configured to circumferentially align pairs of openings 242, 244. In some embodiments, the structural components 332, 334 are configured to transmit shear force between the first and second tubular structures 202, 204 after the first and second tubular structures 202, 204 are coupled to each other.

FIG. 3A shows the first and second tubular structures 202, 204 before they

are coupled to each other. FIG. 3B shows the first and second tubular structures 202, 204 that abut each other, and the indexing elements (pairs of mated structural components 332, 334 circumferentially align the set of first openings 242 at the end of the first tubular structure 202 with the set of first openings 244 at the end of the second tubular structure 204. Each pair of the first openings 242, 244 form a space in which the polymeric connector 220 is to be formed.

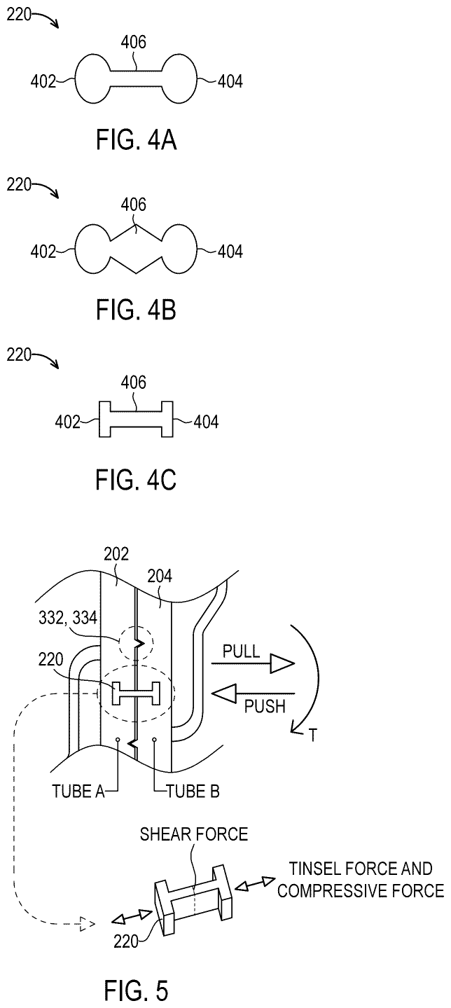

FIG. 4A shows an example of the polymeric connector 220. As shown in the figure, the polymeric connector 220 has a shape that corresponds with the shape of the space formed by the pair of first openings 242, 244. The polymeric connector 220 has a dumbbell shape in the illustrated embodiments. In particular, the polymeric connector 220 has a first enlarged portion 402 at a first end of the polymeric connector, a second enlarged portion 404 at a second end of the polymeric connector, and a relatively smaller center portion 406. In other embodiments, the polymeric connector 220 may have other shapes. For example, in other embodiments, the polymeric connector 220 may have two enlarged portions 402, 404 at opposite ends of the polymeric connector 220, and also an enlarged center portion 406 (FIG. 4B). The enlarged center portion 406 may function as an indexing element configured to align the first tubular structure 202 and the second tubular structure 204 with respect to each other, and/or to transfer force between the first and second tubular structures 202, 204. In further embodiments, the polymeric connector 220 may have an I-shape (FIG. 4C).

In some embodiments, the polymeric connector 220 may be made from polymeric material(s), such as polyurethane, cellulose acetate, mixed esters cellulose, PTFE/polyester, acrylic copolymer, any of other biocompatible polymer, or any combination of the foregoing. Other examples of materials that may be used to make the polymeric connector 220 include: Polyether block amide or PEBA is a thermoplastic elastomer (TPE: tradename of PEBAX® and VESTAMID® E), polyamide (PA: tradename Nylon), Thermoplastic polyurethane (TPU), polyethylene (PE), High Density Polyethylene (HDPE), Low-density polyethylene (LDPE), Polyolefin elastomers (POEs), Styrene-ethylene-butylene-styrene (SEBS), Polyethylene terephthalate (PET or PETE), polylactide (PLA), Fluorinated ethylene propylene (FEP) polymers, or other polymers. The polymeric connector 220 may also include non-polymeric material(s) in some cases. Thus, the term “polymeric connector” is not limited to component that is made solely from a polymeric material, and may refer to a component that is made from one or more polymeric materials, and optionally include one or more non-polymeric materials.

In some cases, the polymeric connector 220 may be made from a material having an elastic modulus that is anywhere from 2 MPa to 2000 MPa. This allows the polymeric connector 220 to be significantly more elastic than the material of the tubular structures 202, 204. Also, in some cases, the polymeric connector 220 allows the tubular member 14 to be more flexible (compared to the solution in which the tubular structures are connected by weld). As a result, the tubular member 14 can navigate through relatively tight bends during use of the catheter 10.

In some cases, the polymeric connector 220 may have a length to allow the tubular member 14 to achieve a desired radius of curvature due to bending of the tubular member 14. A desired radius of curvature for the tubular member 14 may be anywhere from 0.02 inch to 0.16 inch, or from 0.03 inch to 0.10 inch, or from 0.035 inch to 0.08 inch, or from 0.04 inch to 0.06 inch, or 0.055 inch to 0.06 inch, or 0.039 inch +/−0.01 inch. In some cases, the polymeric connector 220 may have a total longitudinal length (measured in a direction that is parallel to the longitudinal axis of the tubular member 14) that is anywhere from 0.2 mm to 2 mm, and more preferably anywhere from 0.2 mm to 1 mm, and even more preferably anywhere from 0.4 mm to 0.8 mm. The opposite anchors of the polymeric connector 220 may each have a longitudinal length (measured in a direction that is parallel to the longitudinal axis of the tubular member 14) that is anywhere from 0.05 mm to 0.15 mm, such as 0.1 mm). In other cases, the opposite anchors of the polymeric connector 220 may each have a longitudinal length that is more than 0.15 mm. Also, the center portion of the polymeric connector 220 extending between the opposite anchors may have a longitudinal length (measured in a direction that is parallel to the longitudinal axis of the tubular member 14) that is anywhere from 0 mm to 5.5 mm, or from 0 mm to 4 mm, or from 0 mm to 3 mm, or from 0 mm to 2 mm, or from 0.4 mm to 1.5 mm, or from 0.4 to 1.0 mm, or from 0.4 to 0.9 mm, or from 0.5 to 0.9 mm. In some cases, when the polymeric connector 220 is implemented more distally (e.g., closer to the distal end of the tubular member 14), the polymeric connector 220 may have a shorter longitudinal length (compared to the scenario when the polymeric connector 220 is implemented more proximally.

Also, in some cases, there may be multiple polymeric connectors 220 disposed circumferentially (at one longitudinal position) between the first and second tubular structures 202, 204. In such cases, the total shear strength V_{T} provided by the polymeric connectors 220 will be equal to the number N of polymeric connectors 220 times the shear strength Vc of each polymeric connector 220. In such cases, the total shear strength V_{T} provided by all of the polymeric connectors 220 may be higher than the shear strength of each of the tubular structures 202, 204.

In some cases, the polymeric connector 220 may be configured to achieve a stiffness profile, and/or a force-displacement profile, that meets the clinical requirement for a particular application.

In some embodiments, each polymeric connector 220 has an uncompressed (neutral) volume, and each polymeric connector 220 is compressible to reach a compressed volume that is less than 50%, less than 40%, less than 30%, less than 20%, less than 10%, or less than 5% of the neutral volume. Also, in some embodiments, each polymeric connector 220 has an unstretched (neutral) volume, and each polymeric connector 220 is stretchable to reach a stretched volume that is more than 105%, more than 110%, more than 120%, more than 130%, more than 140%, or more than 150%, of the neutral volume. In other embodiments, each polymeric connector 220 may be compressible and/or stretchable to reach a strain (compression strain or tensile strain) that is at least 0.1, at least 0.2, at least 0.3, at least 0.4, at least 0.5, wherein a strain is defined as a change in length of the polymeric connector 220 divided by the original length of the polymeric connector 220 (e.g., strain=change in length/original length). In further embodiments, each polymeric connector 220 may be compressible and/or stretchable to reach a strain (compression strain or tensile strain) that is less than 0.1.

Also, in some cases, the polymeric connector 220 may be configured to have a stretch limit that is shorter than a longitudinal length of an indexing element (e.g., the indexing element described with reference to the structural components 332, 334). This allows the indexing element to continue to transfer force (e.g., shear) between the tubular structures 202, 204 even when the polymeric connector 220 is stretched. In other cases, the polymeric connector 220 may be configured to have a stretch limit that is longer than a longitudinal length of the indexing element.

It should be noted that the polymeric connector 220 is not limited to have the configurations shown in the above examples, and that the polymeric connector 220 may have other configurations in other embodiments.

As shown in FIG. 5, in some embodiments, the polymeric connector 220 is configured to transfer one or more forces between the first tubular structure 202 and the second tubular structure 204. The one or more forces may comprise a compressive force, a tensile force, a shear force, a torsional force, or any combination of the foregoing. In some cases, the first and second tubular structures 202, 204 may include indexing element, such as the structural components 332, 334 described herein. In such cases, the indexing element may function to transfer one or more forces, such as axial tension, axial compression, shear, torsion, etc. In other cases, the catheter 10 may not include any indexing element at the tubular structures 202, 204. In such cases, the polymeric connector 220 may function to transfer one or more forces, as described herein.

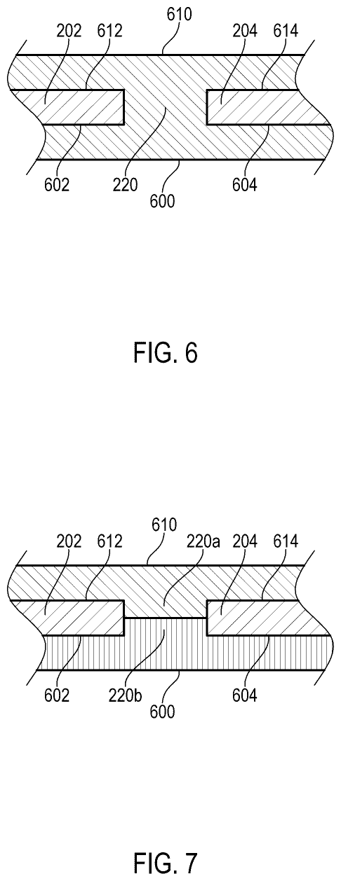

Referring now to FIG. 6, the catheter 10 may optionally further include a liner 600. The liner 600 is on a first inner surface 602 of the first tubular structure 202, and on a second inner surface 604 of the second tubular structure 204. The catheter 10 may optionally further includes a jacket 610. The jacket 610 is on a first outer surface 612 of the first tubular structure 202, and on a second outer surface 614 of the second tubular structure 204. The polymeric connector 220 may be connected to the liner 600 and/or to the jacket 610.

In some cases, the jacket 610 and at least a part of the polymeric connector 220 may be made from a same material. Also, in some cases, the liner 600 and at least a part of the polymeric connector 220 may be made from a same material. In other cases, the polymeric connector 220 may be made from a material that is different from the material of the jacket 610 and/or the material of the liner 600.

The polymeric connector 220 may be connected to the liner 600 by integrally formed with the liner 600, or by forming the polymeric connector 220 on the liner 600. In other cases, the polymeric connector 220 may be connected to the liner 600 via an adhesive (e.g., glue). Similarly, the polymeric connector 220 may be connected to the liner jacket 610 by integrally formed with the jacket 610, or by forming the polymeric connector 220 on of the jacket 610. In other cases, the polymeric connector 220 may be connected to the jacket 610 via an adhesive (e.g., glue). As shown in FIG. 6, in some cases, the polymeric connector 220 may be integral with both the liner 600 and the jacket 610. Such may be accomplished by attaching the first and second tubular structures 202, 204 to the liner 600, or vice versa. Then the material of the polymeric connector 220, in uncured and/or liquid form, is then disposed in the space between the first and second tubular structures 202, 204. The jacket 610 is then disposed over the first and second tubular structures 202, 204, covering the material of the polymeric connector 220. Alternatively, material of the jacket 610 in uncured or liquid form may be placed on the outer surfaces 612, 614 of the respective first and second tubular structures 202, 204. After the material of the polymeric connector 220 (and also the material of the jacket 610 if it has been applied in liquid form) is cured or hardened, the polymeric connector 220 is then formed and is integral with the liner 600 and the jacket 610.

In other cases, the liner 600 is first coupled to the first and second tubular structures 202, 204. Then the polymeric connector 220 in solid form may be placed in the space between the first and second tubular structures 202, 204. The jacket 610 is then disposed around the first and second tubular structures 202, 204. The liner 600, the polymeric connector 220, and the jacket 610 may then be heated to cause opposite ends of the polymeric connector 220 to be respectively fused with the liner 600 and the jacket 610. In some cases, the opposite ends of the polymeric connector 220 may include heat-activated adhesives. In other cases, the polymeric connector 220 may be made from a material with a lower melting point than the material of the liner 600 and the material of the jacket 610. This allows the material of the polymeric connector 220 to be melted while the material is contained between the liner 600 and the jacket 610. After the material of the polymeric connector 220 is solidified or hardened, the polymeric connector 220 is then integral with the liner 600 and the jacket 610.

In some cases, the liner 600 may be provided as a tube, and the first and second tubular structures 202, 204 may be disposed over the tube. The liner 600 may have a surface with a layer of material that is configured to adhere to the surfaces of the first and second tubular structures 202, 204. For example, the layer of material may be an adhesive, or a heat-activated adhesive. After the tube (the liner 600) is heated, the first and second tubular structures 202, 2024 are then attached to the liner 600.

Similarly, the jacket 610 may be provided as a tube, and such tube may be disposed over the first and second tubular structures 202, 204. The jacket 610 may have a surface with a layer of material that is configured to adhere to the surfaces of the first and second tubular structures 202, 204. For example, the layer of material may be an adhesive, or a heat-activated adhesive. After the tube (the jacket 610) is heated, the first and second tubular structures 202, 2024 are then attached to the jacket 610.

In some cases, before the jacket 610 is disposed over the first and second tubular structures 202, 204, material of the polymeric connector 220 (in liquid or solid form) may be disposed into the space between the first and second tubular structures 202, 204. After the material of the polymeric connector 220 is disposed, and after the jacket 610 is placed over the first and second tubular structures 202, 204, heat may then be applied to heat the liner 600 and the jacket 610. The heated liner 600 causes the liner 600 to adhere to the inner surfaces of the first and second tubular structures 202, 204, and also to the material of the polymeric connector 220. Similarly, the heated jacket 610 causes the jacket 610 to adhere to the outer surfaces of the first and second tubular structures 202, 204, and also to the material of the polymeric connector 220.

In further cases, the first and second tubular structures 202, 204 may be dipped into a solution (material). When the solution solidifies, it forms the liner 600 on the inner surfaces 602, 604 of the respective first and second tubular structures 202, 204, forms the polymeric connector 220 between the first and second tubular structures 202, 204, and forms the jacket 610 on the outer surfaces 612, 614 of the respective first and second tubular structures 202, 204.

In other cases, the first and second tubular structures 202, 204 may be sprayed with a solution (material). When the solution solidifies, it forms the liner 600 on the inner surfaces 602, 604 of the respective first and second tubular structures 202, 204, forms the polymeric connector 220 between the first and second tubular structures 202, 204, and forms the jacket 610 on the outer surfaces 612, 614 of the respective first and second tubular structures 202, 204.

In other cases, the act of forming the polymeric connector 220 may comprise laminating a part of the liner 600 and a part of the jacket 610 across the space to create the polymeric connector 220.

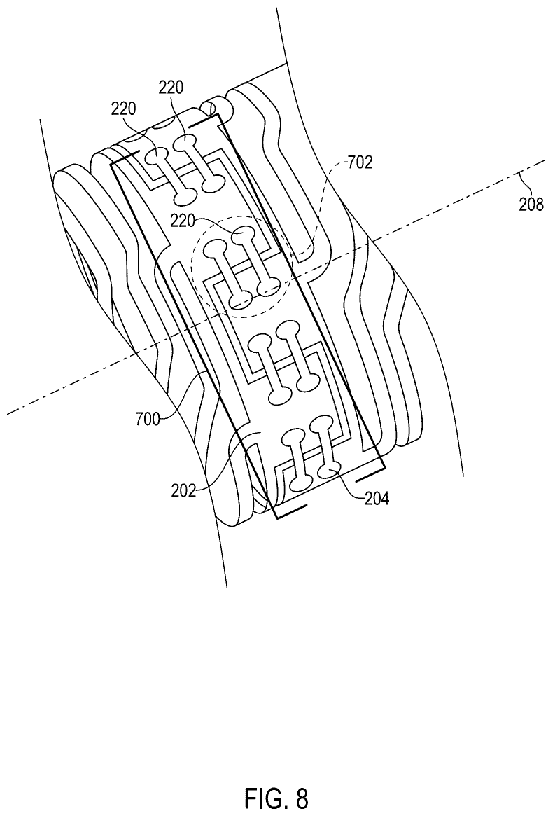

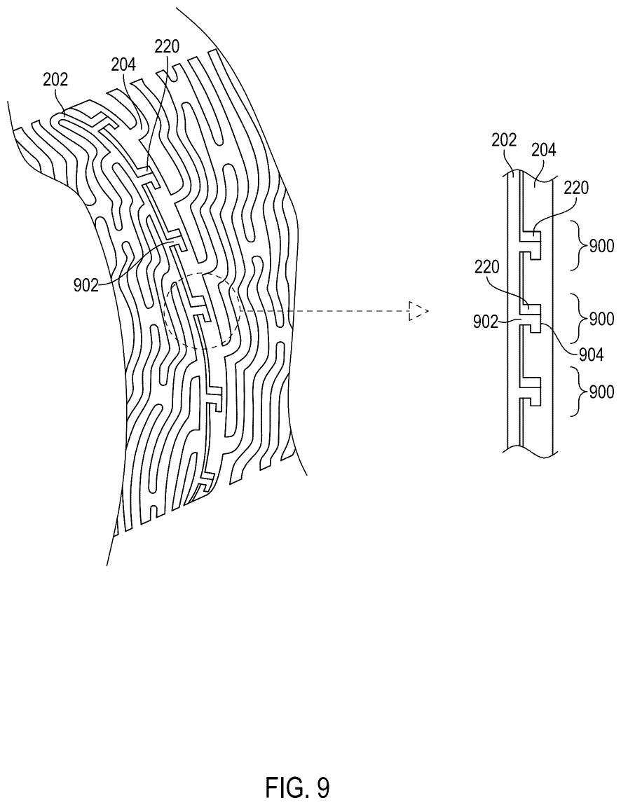

In other cases, the polymeric connector 220 may have a first connector portion 220a, and a second connector portion 220b (FIG. 7). As shown in the figure, the first connector portion 220a and the second connector portion 220b are different respective layers forming the polymeric connector 220. The first connector portion 220a and the second connector portion 220b may be made from the same material, or alternatively, may be made from different respective materials. The first connector portion 220a may be closer to the jacket 610 than to the liner 600, and/or may be coupled directly to the jacket 610. The second connector portion 220b may be closer to the liner 600 than to the jacket 610, and/or may be coupled directly to the liner 600. The seam between the first connector portion 220a and the second connector portion 220b is located in the middle so that the thickness (measure in a direction that is perpendicular to the longitudinal axis 208 of the catheter 10) of the first connector portion 220a is equal to the thickness of the second connector portion 220b. In other cases, the thickness of the first connector portion 220a and the thickness of the second connector portion 220b may be different from each other. For examples, the thickness of the first connector portion 220a may be larger than the thickness of the second connector portion 220b, or the thickness of the first connector portion 220a may be less than the thickness of the second connector portion 220b.

The configuration of the polymeric connector 220 shown in FIG. 7 may be achieved by letting some of the material of the liner 600 flow into the space between the first and second tubular structures 202, 204 when the material is in liquid form, and by letting some of the material of the jacket 610 flow into the space between the first and second tubular structures 202, 204 when the material is in liquid form. The material of the liner 600 and the material of the jacket 610 flow into the space between the first and second tubular structures 202, 204 from opposite ends of the space. Thus, the material of the liner 600 disposed into the space between the first and second tubular structures 202, 204 occupies only a part of the space. Similarly, the material of the jacket 610 disposed into the space between the first and second tubular structures 202, 204 occupies only a part of the space.

In other cases, the catheter 10 may not include the liner 600 and/or the jacket 610. For example, in other cases, the polymeric connectors 220 may not extend beyond the transverse boundaries of the space between the tubular structures 202, 204 (e.g., the inner surfaces of the tubular structures 202, 204, and/or the outer surfaces of the tubular structures 202, 204). In further cases, the polymeric connectors 220 may extend beyond the inner surfaces of the tubular structures 202, 204, and/or beyond the outer surfaces of the tubular structures 202, 204. For example, in other cases, the catheter 10 may include the jacket 610 and the polymeric connectors 220 attached to the jacket 610, wherein the polymeric connectors 220 may extend past the inner surface of the walls of the tubular structure 202 and/or the inner surface of the walls of the tubular structure 204, into the lumen of the tubular member 14. In another example, the catheter 10 may include the liner 600 and the polymeric connector 220 attached to the liner 600, wherein the polymeric connectors 220 may extend past the exterior surface of the walls of the tubular structure 202 and/or the exterior surface of the walls of the tubular structure 204. In some cases, the part of the polymeric connector 220 extending beyond the transverse boundary of the space between the tubular structures 202, 204 may be a bump, an elongated protrusion, a block, or may have a random shape (for example, such as that due to a manufacturing artifact). In still further embodiments, a part of the polymeric connector 220 extending past the interior surface of the tubular structure 202/204 may extend to an inner layer (e.g., the liner 600) disposed on the interior surface of the tubular structure 202/204, wherein the part of the polymeric connector 220 may be separately attached to the inner layer, or may be formed integrally with the inner layer. In further embodiments, a part of the polymeric connector 220 extending past the exterior surface of the tubular structure 202/204 may extend to an outer layer (e.g., the jacket 600) disposed on the exterior surface of the tubular structure 202/204, wherein the part of the polymeric connector 220 may be separately attached to the outer layer, or may be formed integrally with the outer layer. In other embodiments, the polymeric connector 220 may have a first part extending past the exterior surface of the tubular structure 202/204, and a second part extending past the interior surface of the tubular structure 202/204. In such cases, the first part of the polymeric connector 220 may be separately attached to an outer layer (e.g., the jacket 610) disposed on the exterior surface of the tubular structure 202/204, and the second part of the polymeric connector 220 may be separately attached to an inner layer (e.g., the liner 600) disposed on the interior surface of the tubular structure 202/204.

As shown in the above examples, the catheter 10 may have multiple polymeric connectors 220 (like those shown in FIGS. 2-3) that are connected to each other via the liner 600 and/or the jacket 610. In some cases, the liner and/or the jacket together with the polymeric connectors 220 constitute a filler structure that has a tubular or ring-like configuration. The filler structure may include one or more filling portions filling one or more spaces between two adjacent tubular structures.

As shown in FIGS. 6-7, the first tubular structure 202 and the second tubular structure 204 have a same inner diameter and/or a same outer diameter. In other cases, the first tubular structure 202 and the second tubular structure 204 have different respective inner diameters and/or different respective outer diameters.

Also, in some cases, the first tubular structure 202 and the second tubular structure 204 may be made from a same material. In other cases, the first tubular structure 202 and the second tubular structure 204 may be made from different respective materials.

In the above examples, the catheter 10 has been descried as having multiple polymeric connectors 220 (e.g., a polymeric connector and an additional polymeric connector). Each of the polymeric connectors (e.g., the polymeric connector, the additional polymeric connector) is configured to couple the first tubular structure 202 and the second tubular structure 204 to each other, and/or prevent the first tubular structure 202 and the second tubular structure 204 from separating from each other. In other cases, the catheter 10 may have only a single polymeric connector 220 connecting each pair of adjacent tubular structures.

Also, in other cases, the catheter 10 may have more than two tubular structures (e.g., three tubular structures, four tubular structures, etc.). In such cases, each adjacent pair of tubular structures may be connected by one or more polymeric connectors 220.

In the cases of FIGS. 2-3, the polymeric connectors 220 are arranged circumferentially around the longitudinal axis 208 of the catheter 10, and are located in a plane (e.g., a common plane) that is perpendicular to the longitudinal axis 208 of the catheter 10. The polymeric connectors 220 are distributed evenly circumferentially. This has the benefit of creating a catheter body that has the same connection strength on different sides of the catheter. Alternatively or additionally, the polymeric connectors 220 may be in different respective planes that are perpendicular to the longitudinal axis 208 of the catheter 10. For example, as shown in FIG. 8, the catheter 10 has two sets 700, 702 of polymeric connectors 220. The polymeric connectors 220 in the first set 700 are circumferentially disposed around the longitudinal axis 208 of the catheter 10, and are in a first common plane that is perpendicular to the longitudinal axis 208 of the catheter 10. The polymeric connectors 220 in the second set 702 are circumferentially disposed around the longitudinal axis 208 of the catheter 10, and are in a second common plane that is perpendicular to the longitudinal axis 208 of the catheter 10, wherein the second common plane is different from the first common plane.

In the above examples, the polymeric connector 220 has enlarged portions at opposite ends that function as anchors for mechanically connecting the first and second tubular structures 202, 204 to each other. In other cases, the polymeric connector 220 may have different configurations. For example, in other cases, the polymeric connector 220 may have only one enlarged portion that functions as an anchor, or may have multiple enlarged portions. Also, the polymeric connector 220 does not need to “actively” connect the first and second tubular structures 202, 204 to each other, and may be a “passive” connector in some cases. For example, as shown in FIG. 9, the first tubular structure 202 may have a protrusion 902 at the end of the first tubular structure 202, wherein the protrusion 902 is configured for placement in an opening 904 at the end of the second tubular structure 204. In the illustrated example, the protrusion 902 is in a form of a hook that is sized and shaped for insertion into the opening 904. The opening 904 has a hook shape that corresponds with the hook configuration of the protrusion 902. The opening 904 is sized to accommodate at least a part of the protrusion 902. During manufacturing, the first tubular structure 202 is positioned towards the second tubular structure 204, or vice versa, to insert the protrusion 902 into the opening 904. After the protrusion 902 is inserted into the opening 904, the first tubular structure 202 is than rotated about the longitudinal axis relative to the second tubular structure 204, or vice versa, to move the protrusion 902 from one location in the opening 904 to another location in the opening 904, thereby locking the protrusion 902 longitudinally with respect to the opening 904. As shown in the figure, the polymeric connector 220 is located in a part of the opening 904 to prevent the protrusion 902 of the first tubular structure 202 from moving within the opening 904 after the protrusion 902 is inserted into the opening 904. As a result, the protrusion 902 cannot move circumferentially out of the opening 904, causing the first and second tubular structures 202, 204 to stay connected to each other. Thus, as used in this specification, the term “connector” is not limited to a mechanical component that “connects”, and may include a mechanical component (such as a lock, a blocker, a filler, a stopper, etc.) that prevents separation of two items (e.g., two tubular structures).

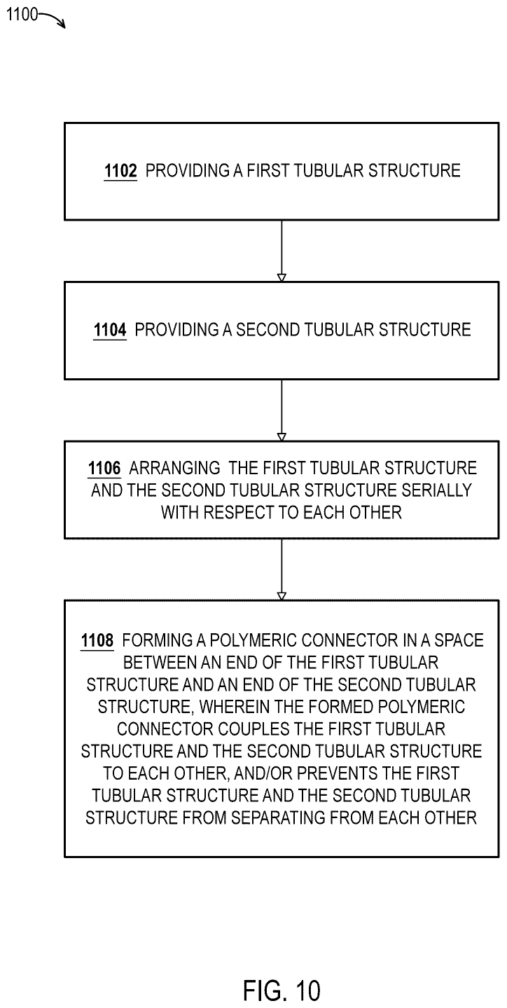

FIG. 10 illustrates a method 1100 of making a part of a catheter. The method 1100 includes: providing a first tubular structure (item 1102); providing a second tubular structure (item 1104); arranging the first tubular structure and the second tubular structure serially with respect to each other (item 1106); and forming a polymeric connector in a space between an end of the first tubular structure and an end of the second tubular structure, wherein the formed polymeric connector couples the first tubular structure and the second tubular structure to each other, and/or prevents the first tubular structure and the second tubular structure from separating from each other (item 1108).

Optionally, in the method 1100, the first tubular structure comprising a first opening at an end of the first tubular structure; the second tubular structure comprising a first opening at an end of the second tubular structure; and the formed polymeric connector comprises a first connector part disposed in the first opening at the end of the first tubular structure, and a second connector part disposed in the first opening at the end of the second tubular structure.

Optionally, in the method 1100, the first opening at the end of the first tubular structure is a laser-cut opening.

Optionally, in the method 1100, the first tubular structure comprises a protrusion at the end of the first tubular structure; the second tubular structure comprises an opening at the end of the second tubular structure, the opening sized to accommodate at least a part of the protrusion; and the formed polymeric connector is located in a part of the opening to prevent the protrusion of the first tubular structure from moving within the opening.

Optionally, in the method 1100, the first tubular structure is a first hypotube made from a metal, an alloy, or a polymer, and wherein the second tubular structure is a second hypotube made from the metal, the alloy, the polymer, another metal, another alloy, or another polymer.

Optionally, in the method 1100, a thickness of the first tubular structure is equal to a thickness of the second tubular structure.

Optionally, in the method 1100, a thickness of the first tubular structure is different from a thickness of the second tubular structure.

Optionally, in the method 1100, a material of the first tubular structure is the same as a material of the second tubular structure.

Optionally, in the method 1100, a material of the first tubular structure is different from a material of the second tubular structure.

Optionally, in the method 1100, the formed polymeric connector has a first enlarged portion at a first end of the polymeric connector, and a second enlarged portion at a second end of the polymeric connector.

Optionally, in the method 1100, the polymeric connector has a dumbbell shape or an I-shape.

Optionally, the method 1100 further includes providing a liner, wherein the liner is on a first inner surface of the first tubular structure, and on a second inner surface of the second tubular structure, and wherein the polymeric connector is connected to the liner.

Optionally, the method 1100 further includes providing a jacket, wherein the jacket is on a first outer surface of the first tubular structure, and on a second outer surface of the second tubular structure, and wherein the polymeric connector is connected to the jacket.

Optionally, the act of providing the liner, the act of providing the jacket, and the act of forming the polymeric connector comprise: placing the liner inside the first and second tubular structures, placing a material of the polymeric connector in the space between the end of the first tubular structure and the end of the second tubular structure, placing the jacket around the first and second tubular structures, and curing the material of the polymeric connector to form the polymeric connector.

Optionally, in the method 1100, the liner is made from a liner material, and the act of forming the polymeric connector comprises letting some of the liner material flow into the space that is between the end of the first tubular structure and the end of the second tubular structure.

Optionally, in the method 1100, the jacket is made from a jacket material, and the act of forming the polymeric connector comprises letting some of the jacket material flow into the space that is between the end of the first tubular structure and the end of the second tubular structure.

Optionally, in the method 1100, the liner material flows from a liner side to a jacket side.

Optionally, in the method 1100, the jacket material flows from a jacket side to a liner side.

Optionally, in the method 1100, the liner material flows from a liner side, and the jacket material flows from a jacket side, and the liner material and the jacket material meets at a location somewhere in the space.

Optionally, the act of providing the liner, the act of providing the jacket, and the act of forming the polymeric connector comprise: dipping the first and second tubular structures into a material to coat the first and second tubular structures with the material, and curing the material to form the liner, the jacket, and the polymeric connector.

Optionally, the act of providing the liner, the act of providing the jacket, and the act of forming the polymeric connector comprise: spraying the first and second tubular structures with a material to coat the first and second tubular structures with the material, and curing the material to form the liner, the jacket, and the polymeric connector.

Optionally, the act of forming the polymeric connector comprise: laminating a part of the liner and a part of the jacket across the space to create the polymeric connector.

Optionally, in the method 1100, the jacket and at least a part of the polymeric connector are made from a same material.

Optionally, in the method 1100, the liner and at least a part of the polymeric connector are made from a same material.

Optionally, in the method 1100, the first tubular structure and the second tubular structure have different respective inner diameters and/or different respective outer diameters.

Optionally, in the method 1100, the first tubular structure and the second tubular structure have a same inner diameter and/or a same outer diameter.

Optionally, in the method 1100, the formed polymeric connector is configured to transfer one or more forces between the first tubular structure and the second tubular structure, and wherein the one or more forces comprise a compressive force, a tensile force, a shear force, a torsional force, or any combination of the foregoing.

Optionally, in the method 1100, the end of the first tubular structure and the end of the second tubular structure are connected to each other without welding, without soldering, and without utilizing any adhesive.

Optionally, the method 1100 further includes forming an additional polymeric connector, wherein the additional polymeric connector is configured to couple the first tubular structure and the second tubular structure to each other, and/or prevent the first tubular structure and the second tubular structure from separating from each other.

Optionally, in the method 1100, the polymeric connector and the additional polymeric connector are arranged circumferentially around the longitudinal axis of the catheter, and are located in a plane that is perpendicular to the longitudinal axis of the catheter.

Optionally, in the method 1100, the polymeric connector and the additional polymeric connector are in different respective planes that are perpendicular to the longitudinal axis of the catheter.

Optionally, in the method 1100, the polymeric connector and the second polymeric connector are parts of a filler structure that has a tubular or ring-like configuration.

Optionally, in the method 1100, each of the first and second tubular structures comprises a structural component, wherein the structural component of the first tubular structure is configured to abut and mate with the structural component of the second tubular structure, and wherein the structural components are configured to transmit shear force.

The polymeric connectors 220 described herein are advantageous because they connect two adjacent tubular structures without welding, without soldering, and without utilizing any adhesive. Accordingly, the tubular structures 202, 204 would not be affected by heat that is generated by welding or soldering. Also, in some cases, the joining of the two tubular structures 202, 204 could be assembled at the same time as the catheter's jacket 610/liner 600 is formed. This offers manufacturing flexibility to assembly a family of catheters with only changing discrete sections of the catheter design. In addition, the polymeric connectors 220 allow assembly of two tubular structures (e.g., hypotubes) regardless of their materials and/or geometries—e.g., the two tubular structures may be made from the same material or from different respective materials, the two tubular structures may have the same thickness or different respective thicknesses, the two tubular structures may have the same internal diameter or differing respective internal diameters (IDs), and/or the two tubular structures may have the same outer diameter or different respective outer diameters (ODs). In some cases, the polymeric connector(s) 220 can withstand compressive, tensile, shear, and/or torsional that is higher than a butt-joint without using weld or glue.

In the above examples, the polymeric connector 220 is described as being in the space between the tubular structures 202, 204. As shown in FIG. 2, the space between the tubular structures 202, 204 accommodating the polymeric connector 220 includes the opening 242 defined by a part of the tubular structure 202 at the end of the tubular structure 202, and the opening 244 defined by a part of the tubular structure 204 at the end of the tubular structure 204. Alternatively or additionally, the space between the tubular structures 202, 204 may include a region that is between a tip of the tubular structure 202 and a tip of the tubular structure 204.

Also, an “end” of a member/structure (e.g., tubular structure 202, tubular structure 204, etc.) may be a segment of the member/structure that is 1/10, 1/20, 1/30, 1/40, or 1/50 of the entire length of the member/structure measured from a tip of the member/structure. In some cases, an “end” of the member/structure may have a longitudinal length of 1 inch, 0.5 inch, 0.4 inch, 0.3 inch, 0.2 inch, 0.1 inch, or less than 0.1 inch.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure. In some cases, the term “about” may refer to a range of values that are within +/−10% of a value. For example, a value of 2 or a value of about 2 may refer to any value that is within the range of 2 +/−10% (=2+/−0.2=1.8 to 2.2).

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Although particular embodiments have been shown and described, it will be understood that it is not intended to limit the claimed inventions to the preferred embodiments, and it will be obvious to those skilled in the art that various changes and modifications (e.g., the dimensions and/or shapes of various parts) may be made without department from the spirit and scope of the claimed inventions. The specification and drawings are, accordingly, to be regarded in an illustrative rather than restrictive sense. The claimed inventions are intended to cover alternatives, modifications, and equivalents.

1. A catheter comprising:
a first tubular structure;
a second tubular structure, wherein the first tubular structure and the second tubular structure are arranged serially with respect to each other along a longitudinal axis of the catheter; and
a polymeric connector located between (1) a part of the first tubular structure at an end of the first tubular structure and (2) a part of the second tubular structure at an end of the second tubular structure;
wherein the polymeric connector is configured to couple the first tubular structure and the second tubular structure to each other, and/or to prevent the first tubular structure and the second tubular structure from separating from each other; and
wherein the first tubular structure comprises a first opening at an end of the first tubular structure, the second tubular structure comprise a first opening at an end of the second tubular structure, and wherein the polymeric connector comprises a first connector part disposed in the first opening at the end of the first tubular structure, and a second connector part disposed in the first opening at the end of the second tubular structure.

2. The catheter of claim 1, wherein the first opening at the end of the first tubular structure is a laser-cut opening.

3. The catheter of claim 1, wherein the first connector part has a curvilinear configuration.

4. The catheter of claim 1, wherein:
the first tubular structure comprises a protrusion at the end of the first tubular structure;
the second tubular structure comprises an opening at the end of the second tubular structure, the opening sized to accommodate at least a part of the protrusion; and
at least a part of the polymeric connector is located in a part of the opening to prevent the protrusion of the first tubular structure from moving within the opening.

5. The catheter of claim 1, wherein the first tubular structure is a first hypotube made from a metal, an alloy, or a polymer, and wherein the second tubular structure is a second hypotube made from the metal, the alloy, the polymer, another metal, another alloy, or another polymer.

6. The catheter of claim 1, further comprising a liner, wherein the liner is on a first inner surface of the first tubular structure, and on a second inner surface of the second tubular structure, and wherein the polymeric connector is connected to the liner.

7. The catheter of claim 6, further comprising a jacket, wherein the jacket is on a first outer surface of the first tubular structure, and on a second outer surface of the second tubular structure, and wherein the polymeric connector is connected to the jacket.

8. The catheter of claim 7, wherein the jacket and at least a part of the polymeric connector are made from a same material.

9. The catheter of claim 1, wherein the first tubular structure and the second tubular structure are made from different respective materials.

10. The catheter of claim 1, wherein the first tubular structure and the second tubular structure have different respective inner diameters and/or different respective outer diameters.

11. The catheter of claim 1, wherein the first tubular structure and the second tubular structure have a same inner diameter and/or a same outer diameter.

12. The catheter of claim 1, wherein the polymeric connector is configured to transfer one or more forces between the first tubular structure and the second tubular structure, and wherein the one or more forces comprise a compressive force, a tensile force, a shear force, a torsional force, or any combination of the foregoing.

13. The catheter of claim 1, wherein the end of the first tubular structure and the end of the second tubular structure are connected to each other without welding, without soldering, and without adhesive.

14. The catheter of claim 1, wherein the first connector part of the polymeric connector has a first enlarged portion at a first end of the polymeric connector, and wherein the second connector part of the polymeric connector has a second enlarged portion at a second end of the polymeric connector.

15. The catheter of claim 1, further comprising an additional polymeric connector, wherein the additional polymeric connector is configured to couple the first tubular structure and the second tubular structure to each other, and/or prevent the first tubular structure and the second tubular structure from separating from each other.

16. The catheter of claim 15, wherein the polymeric connector and the additional polymeric connector are arranged circumferentially around the longitudinal axis of the catheter, and are located in a plane that is perpendicular to the longitudinal axis of the catheter.

17. The catheter of claim 15, wherein the polymeric connector and the additional polymeric connector are in different respective planes that are perpendicular to the longitudinal axis of the catheter.

18. The catheter of claim 15, wherein the polymeric connector and the second polymeric connector are parts of a filler structure that has a tubular or ring-like configuration.

19. The catheter of claim 1, wherein each of the first and second tubular structures comprises a structural component, wherein the structural component of the first tubular structure is configured to abut and mate with the structural component of the second tubular structure, and wherein the structural components are configured to transmit shear force.

20. A method of making a part of a catheter, the method comprising:
providing a first tubular structure;
providing a second tubular structure;
arranging the first tubular structure and the second tubular structure serially with respect to each other; and
forming a polymeric connector in a space between an end of the first tubular structure and an end of the second tubular structure, wherein the formed polymeric connector couples the first tubular structure and the second tubular structure to each other, and/or prevents the first tubular structure and the second tubular structure from separating from each other;
wherein the first tubular structure comprises a first opening at an end of the first tubular structure, the second tubular structure comprise a first opening at an end of the second tubular structure, and wherein the formed polymeric connector comprises a first connector part disposed in the first opening at the end of the first tubular structure, and a second connector part disposed in the first opening at the end of the second tubular structure.
